(19) United States
(12) Patent Application Publication
(10) Pub. No.: US 2016/0206224 A1
(43) Pub. Date: Jul. 21, 2016

(54) ECG ELECTRODE SNAP CONNECTOR AND ASSOCIATED METHODS

(71) Applicant: MEDICOMP, INC., Melbourne, FL (US)

(72) Inventor: Monte Marek, Palm Bay, FL (US)

(73) Assignee: MEDICOMP, INC., Melbourne, FL (US)

(21) Appl. No.: 14/600,939

(22) Filed: Jan. 20, 2015

(51) Int. Cl. A61B 5/0416; A61B 5/00

(52) U.S. Cl. CPC A61B 5/0416; A61B 5/7246; A61B 5/0022

(57) ABSTRACT

A connector includes a housing, a female snap connector member carried by the housing and configured to mechanically and electrically connect to a male snap connector member of an electrode, a three-axis accelerometer carried by the housing and configured to sense proper acceleration of the connector, and a microprocessor in electrical communication with the snap connector and with the accelerometer. The microprocessor is configured to receive cardiac activity data from the electrode, to receive proper acceleration data from the accelerometer, and to correlate the cardiac activity data to the proper acceleration data to define processed data.

### FIELD OF THE INVENTION

The present invention relates to systems and methods for simultaneously tracking ECG (electrocardiogram) data and movement/acceleration data using a common sensor housing.

### BACKGROUND

In conventional ECG signal measurement, disposable adhesive electrode patches are affixed to the surface of the skin and electrocardiograph lead wires are attached to these electrodes. There are many ways of attaching the lead wires to these electrodes with the most common being the use of parallel spring snaps.

While taking ECG signal measurements, stray signals, which do not relate to the heart signal, that is artifacts, can occur. One of the major causes of artifacts is the movement of the patient's body during the signal measurements, which can produce an electrical signal not related to the heart's electrical signal. The occurrence of artifacts can lead to incorrect ECG data and can be difficult and time consuming to identify and remove from a set of data.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to collecting motion data, which corresponds to a location at which an ECG reading is taken. This may advantageously allow enhanced diagnosis and a reduction in artifacts.

These and other objects, features and advantages according to the present invention are provided by a connector that includes a housing, a female snap connector member carried by the housing and configured to mechanically and electrically connect to a male snap connector member of an electrode, a three-axis accelerometer carried by the housing and configured to sense proper acceleration of the connector, and a microprocessor in electrical communication with the snap connector and with the accelerometer. The microprocessor may be configured to receive cardiac activity data from the electrode, to receive proper acceleration data from the accelerometer, and to correlate the cardiac activity data to the proper acceleration data to define processed data.

The connector may further include a computer readable non-transitory storage medium carried by the housing. The microprocessor may be configured to store the cardiac activity data, the proper acceleration data, and/or the processed data to the storage medium.

The connector may also include a lead wire that may be at least partially carried by the housing and in data communication with the microprocessor and with a computing system. The lead wire may be configured to electronically convey the cardiac activity data, proper acceleration data, and/or the processed data to the computing system.

The connector may also include a wireless transmitter in data communication with the microprocessor. The wireless transmitter may be configured to wirelessly transmit at least one of the cardiac activity data, proper acceleration data, and the processed data to a wireless receiver. The wireless receiver may also be configured to electronically convey the cardiac activity data, proper acceleration data, and/or the processed data to a computing system.

The connector may also include an amplifier and an analog-to-digital converter. The snap connector may be configured to pass the cardiac activity data in analog format to the amplifier. The amplifier may be configured to pass the cardiac activity data in amplified form to the analog-to-digital converter. Similarly, the analog-to-digital converter may be configured to pass the cardiac activity data in digital format to the microprocessor. The accelerometer may be configured to pass the proper acceleration data in digital format to the microprocessor and may be of a capacitive type. Further, the electrode may be an Association for the Advancement of Medical Instrumentation (AAMI) and/or an American National Standards Institute (ANSI) standard disposable ECG electrode.

Another aspect of the invention relates to a retrofit heart rate monitoring system and may include a computing system and a connector as described above. The cardiac activity data, proper acceleration data, and/or the processed data may be transmitted by the monitoring device to the computing system. The accelerometer of the connector may be configured to pass the proper acceleration data in digital format to the microprocessor. The computing system may be configured to determine whether the connector is detached from the electrode using the proper acceleration data and the processed data. The computing system may be configured to reduce movement artifacts using the processed data and may also be configured to detect respiratory distress, sleep disturbance, and/or cardiovascular morbidity using the processed data.

Another aspect of the invention relates to a method of retrofitting an ECG monitoring system using a connector including a female snap connector member, a three-axis accelerometer, and a microprocessor in electrical communication with the snap connector and with the accelerometer. The method may include mechanically and electrically connecting the female snap connector member of the connector to a male snap connector member of an electrode. The method may also include detecting cardiac activity of a patient using the electrode. The method may further include detecting proper acceleration of the connector using the accelerometer. The method may still further include transmitting cardiac activity data to the microprocessor from the electrode and transmitting proper acceleration data to the microprocessor from the accelerometer. The method may also include correlating, using the microprocessor, the cardiac activity data and the proper acceleration data to define processed data.

### BRIEF DESCRIPTION OF THE DRAWINGS

FIG. 1 is a perspective view of an ECG electrode snap connector having portions cut away according to embodiment of the present invention.

FIG. 2 is a cross sectional view of the ECG electrode snap connector illustrated in FIG. 1 and taken through line A-A in FIG. 1.

FIG. 3 is top view of the ECG electrode snap connector illustrated in FIG. 1 showing components carried by a housing of the ECG electrode snap connector.

FIG. 4 is a perspective view of a circuit board carried by the housing of the ECG electrode snap connector illustrated in FIG. 1.

FIG. 5 is a schematic a block diagram of the ECG electrode snap connector according to an embodiment of the present invention.

FIG. 6 is a wiring layout of the ECG electrode snap connector according to an embodiment of the present invention.

FIG. 7 is an environmental view of a plurality of ECG electrode snap connectors according to an embodiment of the present invention in interaction with a patient.

FIG. 8 illustrates an exemplary computer system.

FIG. 9 is an exemplary method associated with the system of FIG. 5.

FIGS. 10 and 11 are additional exemplary methods associated with the system of FIG. 5.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

Additionally, quantitative qualifying terms such as “about,” “approximately,” and “near” and other terms are used, in general, to mean that the referred to object, characteristic, or quality is within a range or comprises a sufficiently similar characteristic so as to achieve the intended function or result of the invention.

Throughout this specification, the invention may be referred to as an electrocardiogram (ECG) snap connector, a snap connector, a connector, a retrofit ECG snap connector, a retrofit snap connector, and/or a retrofit connector. These are not meant to refer to different inventions, but rather, are all embodiments of the present invention.

An electrocardiogram (ECG) is a non-invasive diagnostic tool used to record electrical activity of the heart. This is done by measuring potential difference between several electrodes which are placed on the skin at predefined points of the human body. One cycle of the ECG may represent a depolarization/repolarization of the atrium and the ventricle, which occurs for every heartbeat. This information can be useful in diagnosing and monitoring subjects.

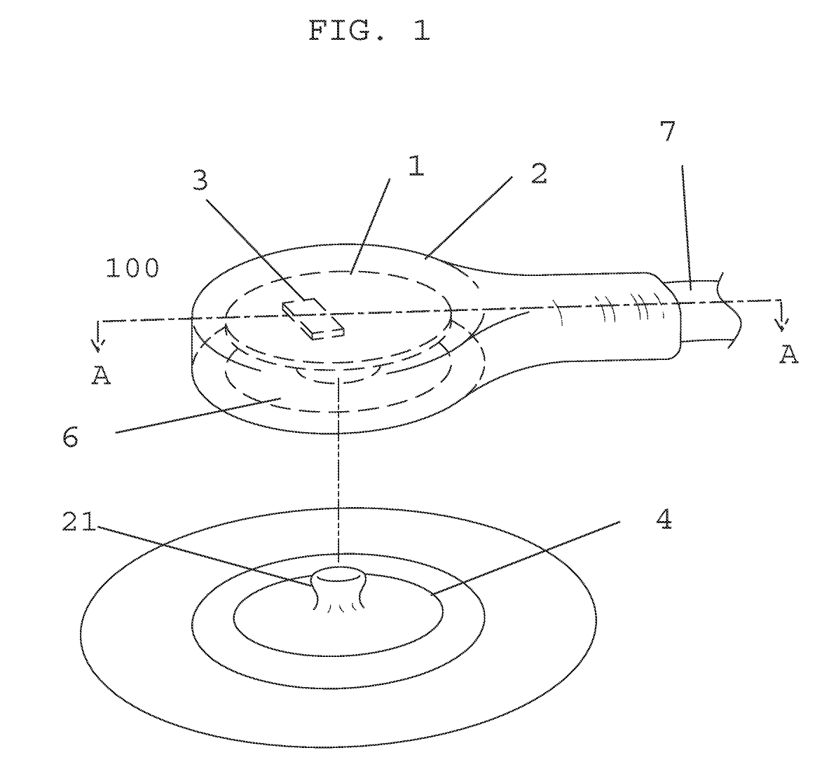

FIGS. 1-10 illustrate exemplary embodiments of the ECG electrode snap connector, a retrofit heartrate monitoring system, and a method for retrofitting an ECG monitoring system with an ECG electrode snap connector according to the present invention.

FIG. 1 illustrates an exemplary sensor assembly 100 and an electrode 4 onto which the sensor assembly 100 may be connected. The electrode 4 may be a commercially available electrode such as those used to attach to a patient's skin. The illustrated electrode 4 may, for example, have an adhesive portion along a bottom portion thereof to allow for the electrode to be readily attached to the patient's skin. Of course, electrodes 4 of any suitable type may be used as desired depending on the specific use.

The sensor assembly 100 may include a circuit board 1 connected to a movement sensor 3. The movement sensor 3 may include an integrated circuit micro-electro-mechanical sensor (MEMS) such as an accelerometer 3, or other movement/location detecting sensor. Of course, the accelerometer/motion sensor 3 may be any type of appropriate sensor and is not limited to a MEMS device. Those skilled in the art will appreciate that any device capable of detecting motion may be suitable for the movement sensor and is intended to be included in the scope of this invention.

In some embodiments, the accelerometer 3 may be a three-axis accelerometer, which may be suitable to detect motion and/or acceleration in any direction, tilt, etc. This may allow the accelerometer 3 to detect the proper acceleration of the sensor assembly 100, and thus the electrode 4, when connected. Furthermore, the accelerometer 3 may be configured to detect the proper acceleration of the sensor assembly 100 regardless of the orientation of the accelerometer 3, objectively or relative to any point of reference, such as the electrode 4 or the person to whom the electrode 4 is attached.

Proper acceleration is physical acceleration (i.e., measurable acceleration as by an accelerometer) experienced by an object. It is thus acceleration relative to a free-fall, or inertial, observer who is momentarily at rest relative to the object being measured. Gravitation therefore does not cause proper acceleration, since gravity acts upon the inertial observer that any proper acceleration must depart from (accelerate from). A corollary is that all inertial observers always have a proper acceleration of zero. Thus, proper acceleration may omit the acceleration of gravity.

Thus, proper acceleration may be thought of as the acceleration detected by the accelerometer 3. In particular, the proper acceleration is the acceleration experienced in relationship to the inertial reference point at which the reading is taken. This is useful as it describes the acceleration experienced by the sensor assembly 100 at a point in time. This information can then be used to determine if the sensor assembly 100 is moving in a manner which is likely to cause a movement artifact in the signal data detected.

In addition, by having a three-axis accelerometer 3, the movement of the sensor assembly 100 may be detected in any direction, so as to ensure that the proper acceleration is detected for all directional movement. A three-axis accelerometer 3 also allows the detection of the tilt of the sensor assembly 100, so as to detect the attitude of the sensor assembly 100 and/or any rotation the sensor assembly 100 is experiencing. This information can be further used to determine if the subject is sitting, lying down, standing, running, etc., as the accelerometer 3 may provide a more complete picture of what the sensor assembly 100 is experiencing, as compared with using single-axis accelerometers which only detect movement along one axis.

The circuit board 1 and the accelerometer 3 may be housed, or partially housed, in a housing 2. The housing may, for example, be provided by an overmold 2. Those skilled in the art will appreciate that although the housing of the ECG electrode snap connector according to embodiments of the present invention is illustrated as an overmold housing, the housing 2 may also be provided by several other types of housings, i.e., snap housing, integrally molded housing, or any other type of housing that may be suitable for containing the various components of the sensor assembly 100. With the above in mind, however, the sensor assembly 100 may be overmolded in a plastic snap overmold 2 so as to resemble a conventional ECG electrode snap connector. The sensor assembly 100 (e.g., a snap connector assembly) may then be able to connect onto a commercially available electrode 4 attached to the patient's skin through the use of electrode connector 6 (e.g., a metal snap).

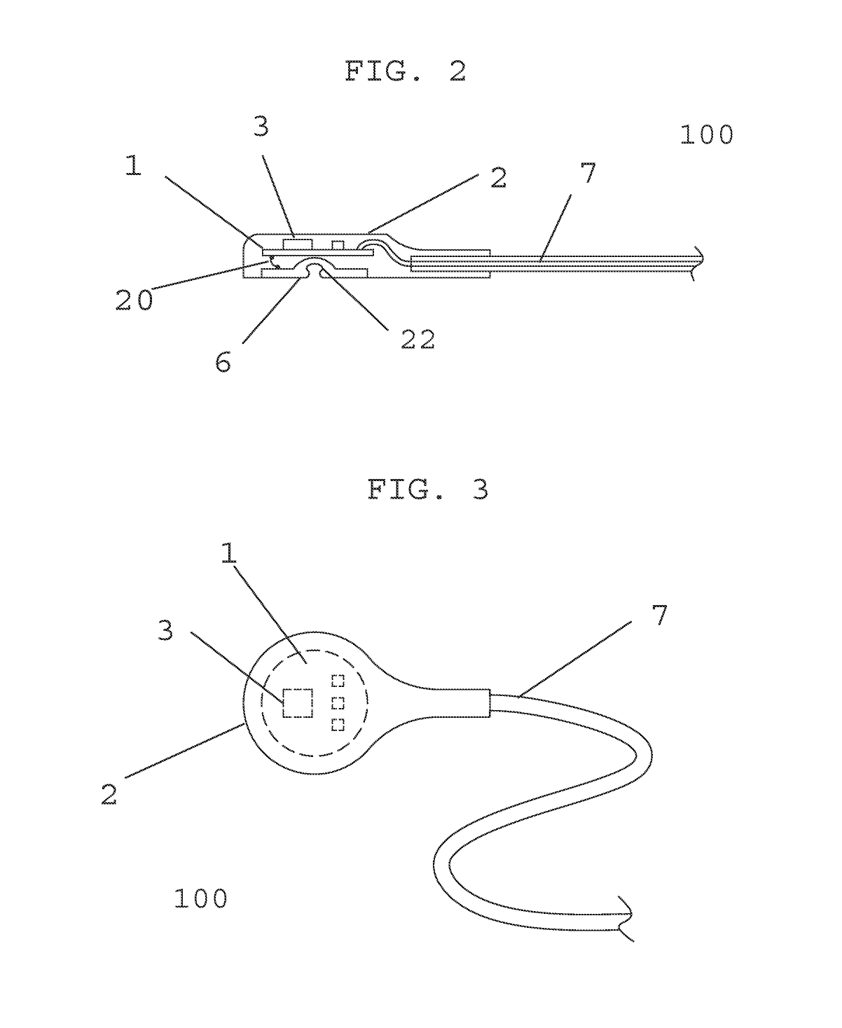

As illustrated in FIGS. 1 and 2, the electrode connector 6 of the ECG electrode snap connector (sensor assembly 100) may be a female connection 22 adapted to engage a male connection portion 21 of the electrode 4. In some embodiments, the female connection 22 may include parallel spring snaps. Of course, any other connection system/method may be configured to attach to standard electrodes 4 is contemplated and included within the scope of the invention. Both the male connection portion 21 of electrode 4, and the female connection 22 of sensor assembly may be formed of a conductive material, such as metal. Thus, the electrode connector 6 may connect to the electrode 4 physically, thereby establishing an electrical connection therebetween.

Furthermore, the connection between the sensor assembly 100 and the electrode 4 may be configured to eliminate or minimize relative motion between the sensor assembly 100, and by extension the accelerometer 3, and the electrode 4. This may eliminate or reduce the chance that the electrode 4 will experience movement that is undetected by the accelerometer 3, or that the accelerometer 3 will experience and detect movement that is not experienced by the electrode 4, thus providing a false indication of movement. This may be accomplished by using a more stable connection method and/or stronger spring clamps on the female connection 22. It is also contemplated that housing 2 may include positioning nubs or ridges around female connection 22 so as to ensure stable contact of the housing 2 with the electrode 4.

In addition, the location of the accelerometer 3 may be positioned close to the female connector (e.g., just over the female connector). By so doing, any relative movement between the electrode 4 and the sensor assembly 100 may be minimized. For instance, if the sensor assembly 100 is rotated quickly or wobbles on the electrode 4, having the accelerometer 3 located above the female connection 22 may eliminate large movements caused by the moment force on the sensor assembly 100. In other words, if the sensor assembly 100 moves, then the accelerometer 3 will not be moved far relative to the female connection 22, as opposed to if the accelerometer 3 were located on a distal end of the sensor assembly 100 (e.g., by the lead 7 where the distance moved by the accelerometer would be greater than above the female connection 22 upon rotation of the sensor assembly 100).

The circuit board 1 may be positioned above the electrode connector 6 as shown, or be located in some other position. The circuit board 1 may communicate with the electrode connector 6 so as to receive a signal therefrom. The method of communication between the circuit board 1 and the electrode connector 6 is not particularly limited and may include, for instance, an electrical connection (e.g., wire 20), an electromagnetic coupling connection, wireless communication, etc. As noted above, the electrode connector 6 may be configured so as to physically and electrically interface with the connection portion 21 of the electrode 4.

The sensor assembly 100 may also be configured so as to connect to a lead wire 7. For example, an ECG signal, or some other signal type, may be conveyed from the electrode 4, through the electrode connector 6, and into the lead wire 7. Additionally, the signal from the electrode connector 6 may pass through the circuit board 1 prior to reaching the lead wire 7. In some embodiments, the lead wire 7 may be fixedly attached so as to be non-removably connected to the circuit board 1 and/or the sensor assembly 100. This allows the monitoring of ECG signals from a patient 14. In some embodiments, the lead wire 7 may be removably attached such that a replacement lead wire 7 may be connected to the sensor assembly 100. The lead wire 7 may also be configured to supply power to sensor assembly 100.

The circuit board 1 may also contain all the necessary support components for operation of the accelerometer 3 such as a signal processor or memory buffer. The circuit board 1 may include connection points for the attachment of signal wires associated with the accelerometer 3 which may be configured to facilitate transmission of a signal from the accelerometer 3 to the lead wire 7. It is also contemplated that the accelerometer 3 may connect directly to the lead wire 7. Optionally, the circuit board 1 may establish communication with the lead 7 through the use of a connector. The connector is not particularly limited and may include, for instance, a female pin connector which can attach to a male connector in the end of lead 7. This may allow the lead 7 to be disconnected from the sensor assembly 100.

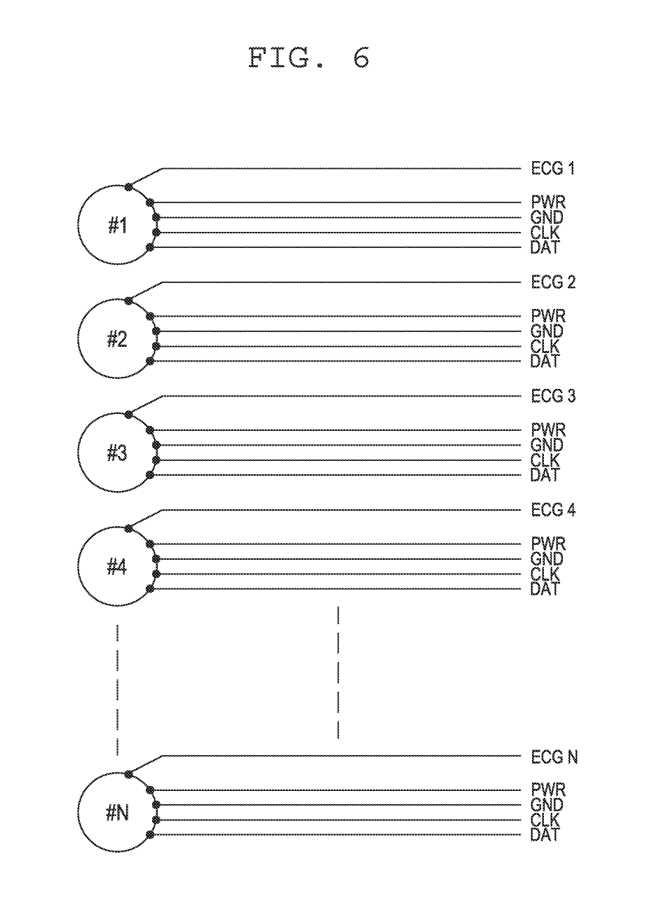

The above features may allow motion data from the accelerometer 3 to be simultaneously captured along with the ECG signal from the electrode 4. Information from the accelerometer 3 may be transmitted through the lead wire 7 to acquisition and processing electronics 15. The processing electronics 15 will be discussed in greater detail below with reference to FIG. 5.

It is also possible for the information from the accelerometer 3 to be transmitted through another wire, or wirelessly, to the processing electronics 15, and/or to some other signal receiving device such as a personal computer. Any wireless communication standard as may be known in the art, including, but not limited to, Zigbee, Bluetooth, Wi-Fi, and any other wireless communication standard is contemplated and included within the scope of the invention. Furthermore, it is contemplated that the ECG signal may be similarly transmitted to the processing electronics 15 and/or another device wirelessly.

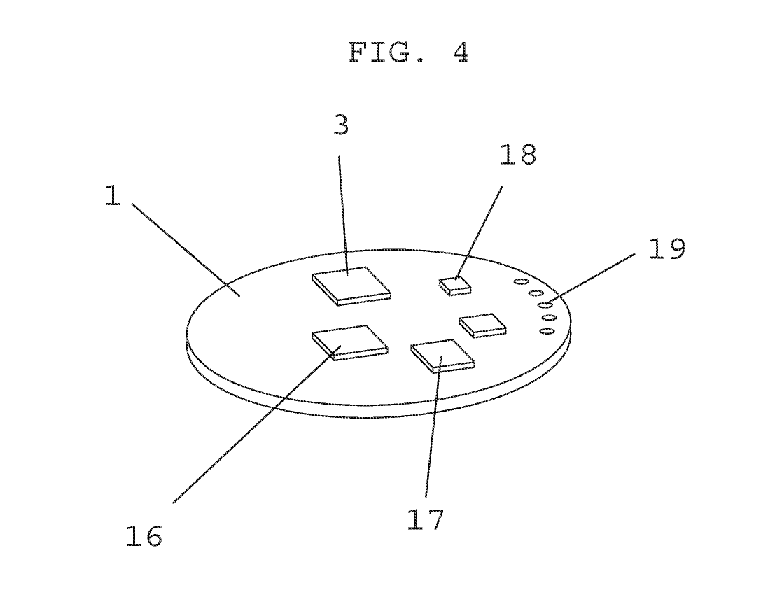

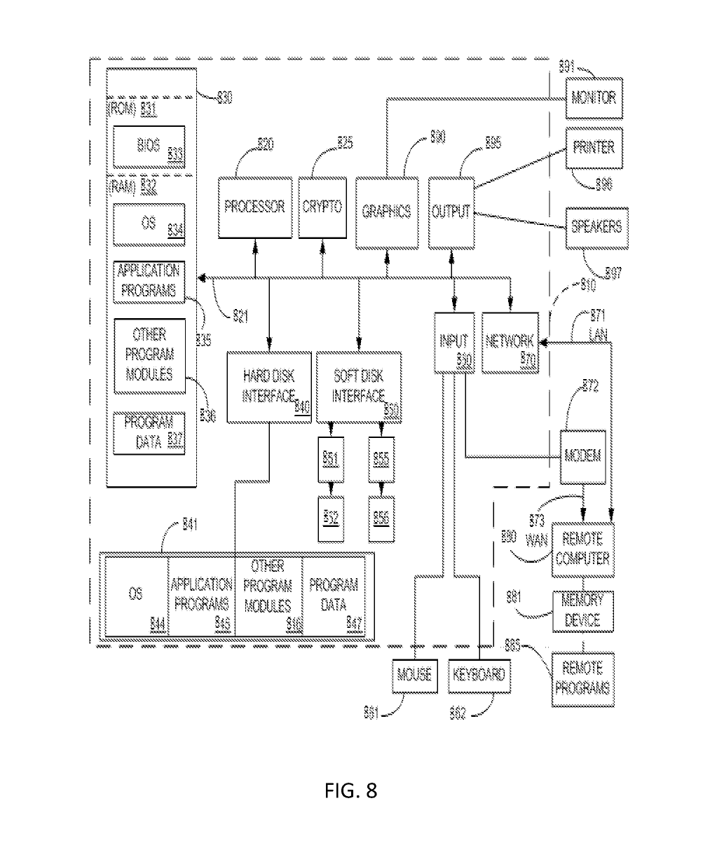

Referring now to FIG. 2, which shows a side view, and FIG. 3, which shows a top view, of the internal construction of an exemplary embodiment of the invention, additional details of the sensor assembly 100 will now be discussed. The circuit board 1 containing the accelerometer 3 may be positioned generally adjacent to and/or above the electrode connector 6 and covered with an overmold compound to create the final overmold assembly 2. The lead wire 7 may contain signal wires, which may be configured to attach to the circuit board 1. The lead wire 7 may additionally connect to the processing electronics 15, as shown in FIG. 7. It is also contemplated that the lead wire 7 may attach to an adaptor or quick disconnect, which may, in turn, connect to processing electronics 15.

Additional signals may also be detected or generated by the circuit board 1. The types of additional signals which can be detected or generated is not particularly limited and the sensor(s) required to detect a given signal may be integrated into the overmold assembly 2. For example, the circuit board 1 may generate and transmit a clock signal, identification information, etc. Similarly, the sensor assembly may include sensors to detect a patient temperature, a local temperature, patient oxygen levels, patient conductivity, etc. Furthermore, the additional signals may be similarly transmitted via the lead wire 7 or any other wired or wireless communication method of which the sensor assembly 100 is configured to support.

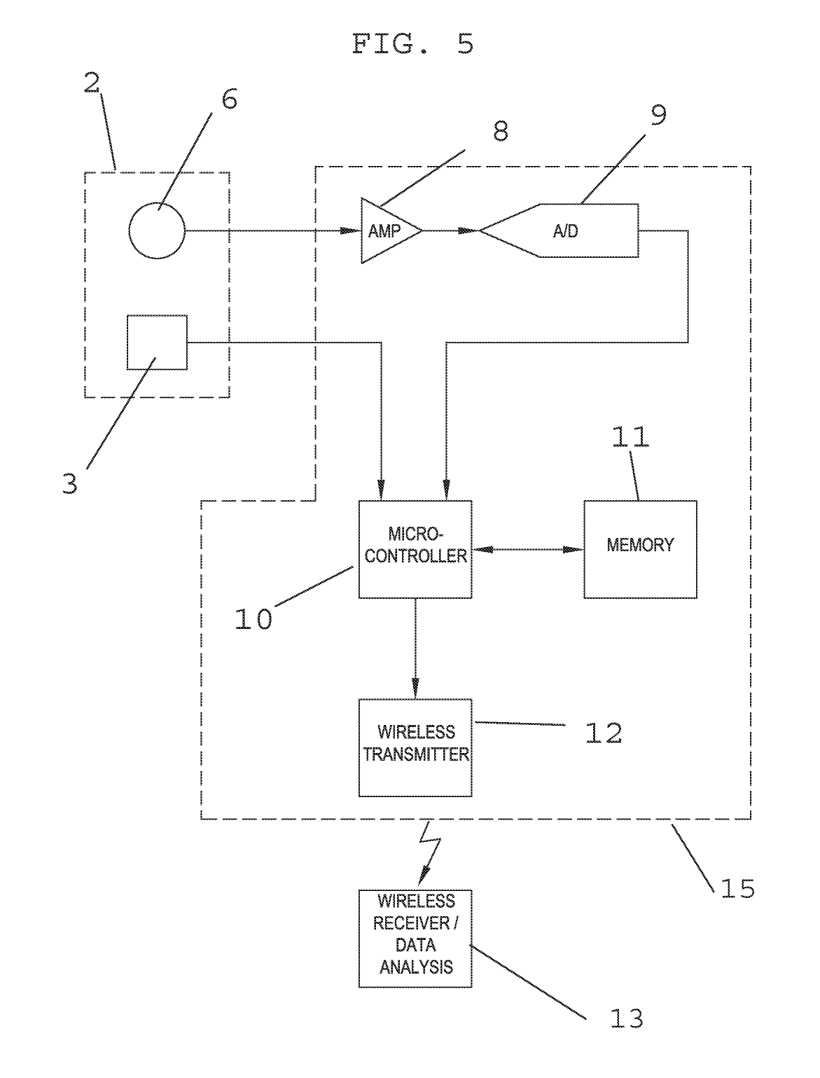

Referring now to FIG. 4, a further arrangement of the circuit board 1 mounted within the ECG snap overmold containing additional motion sensors or other medical sensors, temperature sensors, etc, is presented. As noted above, the circuit board 1 may include an accelerometer 3. Optionally, the circuit board 1 may further include additional sensors 16 and 17. Additional sensors 16 and 17 may include temperature sensors, oxygen sensors, an electrical signal generator and detector to evaluate resistance and/or signal quality between two or more sensor assemblies 100, or any other biological or environmental data.

Furthermore, the circuit board 1 may additionally include support components 18, such as a power circuit having a capacitor and/or battery along with wireless transmitters and/or receivers to send data to the processing electronics 15. This may allow the sensor assembly 100 to be wireless. The circuit board 1 may also include one or more wire attachment structures 19, such as holes or passageways, configured to facilitate the attachment of a wire thereto. However, other means of wire attachment are contemplated and included within the scope of the invention, including, but not limited to, an electrical connector, weld, solder, etc.

Referring now to FIG. 5, an exemplary embodiment of circuitry for the electrical processing of the signals from sensor assembly 100 is presented. Similar to that which has been described above, the sensor assembly 100 may include an ECG snap connector assembly having an overmold housing (snap overmold) 2, an accelerometer 3, and an ECG snap electrode connector 6. An ECG signal from the electrode connector 6 may be conducted into the amplifier 8 and sampled by an analog to digital converter (A/D) 9. A microprocessor or microcontroller integrated circuit (IC) 10 may read the data from the ND 9. The microcontroller 10 can also read digital data, directly or indirectly, from the accelerometer 3 and/or any other sensor of the sensor assembly 100. This data can be processed and synchronized so that movement of the sensor assembly 100 can be correlated with the ECG signal if desired. In the configuration shown in FIG. 5, data may also be stored in a memory 11 and transferred wirelessly from the wireless transmitter 12 to a wireless receiver included in data analysis electronics 13 associated with another computerized device for further processing and presentation. The encasement of processing electronics 15, for instance the electronics doing the ECG and motion sensor signal processing, could be portable and patient worn, as shown in FIG. 7, or encompassed within other physiological patient monitoring equipment in a hospital bedside environment, for example.

In some embodiments, some or all of the electronics associated with the processing electronics 15 may be housed in the sensor assembly 100 instead of a separate enclosure. For instance, the amplifier 8, and possibly the ND 9, may be placed in the sensor assembly 100 to enhance transmission of the ECG signal from the sensor assembly 100. Thus, in some embodiments, additional sensors 16 and 17 may include an amplifier and A/D, respectively, as well as a wireless communication transmitter. Therefore, in some embodiments, the separate processing electronics 15 may be omitted and the data transferred from the sensor assembly 100 to data analysis electronics 13.

While FIG. 5 depicts an exemplary device having memory 11, this is not required in all embodiments. Memory 11 may be used to store instructions for the micro-controller 10, buffer or store data from the accelerometer 3 and/or the electrode connector 6, etc. For example, the memory 11 may be used to store the cardiac activity data, the proper acceleration data, and the processed data.

A wired connection may be used in place of, or in addition to, the wireless transmitter 12 and the wireless receiver included in the data analysis electronics 13. It is also contemplated that the data analysis portion electronics 13 may be formed integrally with the acquisition and processing electronics 15, or may be connected through a wired connection. Indeed, it is possible for the microprocessor 10 to run the acquisition and processing electronics 15, as well as the data analysis electronics 13.

In some embodiments, the signal from the accelerometer 3 may be sent to the processing electronics 15 and then to data analysis electronics 13, and the signal from the electrode connector 6 may be sent to other separate processing electronics, such as a legacy ECG reader 60 (e.g., an ECG reader which does not analyze motion data). This may allow legacy ECG readers to be used with equipment to gather the information from the accelerometer 3 and use this information to analyze the ECG data.

Thus, in some embodiments both the accelerometer data and the ECG data may be transmitted to the data analysis electronics 13, which may be a computer or dedicated ECG reader which can take into account motion information.

In other embodiments, the accelerometer data may be transmitted to the data analysis electronics 13 and the ECG data may be transmitted to the legacy ECG reader 60. The ECG information may then be transferred to the data analysis electronics 13 from the legacy ECG reader 60. Alternatively, the ECG data may be sent to both the legacy ECG reader 60 and the data analysis electronics 13 in parallel.

It is also contemplated that in some embodiments the ECG data and accelerometer data may be sent to the data analysis electronics 13. The data analysis electronics 13 may then remove the ECG data which is caused by movement artifacts and transmit the adjusted ECG data to the legacy ECG reader 60.

For some embodiments, it is contemplated that signals from the electrode 4 and the accelerometer 3 may be sent on the same wire though frequency modulation or any other suitable method. Alternatively, the signals from the electrode 4 and the accelerometer 3 may be sent through different wires or wirelessly.

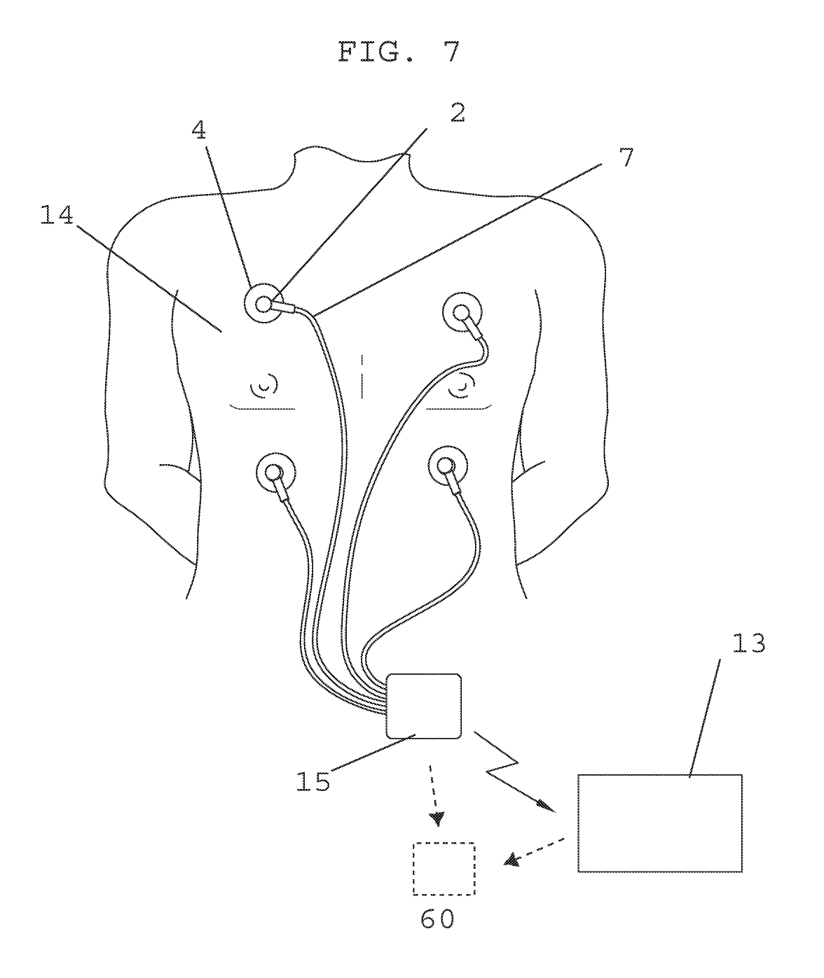

FIG. 6 shows an embodiment of the wiring of an array of sensors, #1 through N. Any number of sensors from 1 to N may be accommodated, one for each ECG electrode. The overmold assemblies 2 in FIG. 7 may each include five signals being conveyed. ECGn, where n is the sensor number, is unique for each sensor and is the conventional ECG signal picked up from the disposable electrode. PWR, GND, DAT, and CLK are power, ground, data, and clock signals respectively and are shared by each motion sensor.

FIG. 7 illustrates an exemplary embodiment having an arrangement of electrodes 4 on the torso of a patient 14. Sensor assemblies 100 are connected to the electrodes 4 through electrode connector 6 and to the acquisition and processing electronics 15 through leads 7. The acquisition and processing electronics 15 may send the collected data to the data analysis electronics 13 through the use of a wireless transmitter and a wireless receiver.

As illustrated in FIG. 7, this embodiment of the invention may make use of multiple electrodes 4 connected to sensor assemblies 100 on the same patient 14. Thus, multiple channels of ECG signals and movement data can be simultaneously acquired from the patient so as to give information on the position and movement of the patient 14 which may be correlated with the ECG signals from electrodes 4. Through the use of position/acceleration data in conjunction with ECG readings, more advanced analysis of the patient may be achieved. While four sensor assemblies 100 attached to four electrodes 4 are depicted, any number of sensor assemblies 100 are contemplated and included within the scope of the invention.

For instance, ECG devices suffer from motion induced artifacts. However, the use of motion data can allow a reduction in artifacts in the data. In one embodiment, if the accelerometer 3 detects questionable movement, for example movement over a certain threshold level (e.g., acceleration over a certain amount, movement greater than a certain amount, average amount, etc.) at a particular electrode 4, then the signal from the electrode 4 at that time can be eliminated as a valid data source. This allows possible motion induced artifacts to be omitted from the data preemptively so as to not require a cumbersome analysis of the ECG data to try to determine if an artifact is present and then removing the article from the data set.

The threshold level of movement may be adjustable according to the type and level of activity of the patient 14 anticipated while the ECG signal is being collected. For example, a first threshold level may be set for when the patient 14 is anticipated to be resting, and a second threshold level may be set for when the patient 14 is anticipated to be active, for instance, during a cardiac stress test. Thus, differing levels of tolerance for the severity of artifacting in an ECG signal may be reflected in adjusting the threshold level of movement.

The combined sources of data can allow other information to be used in conjunction with the ECG data. That is, it can be determined if the patient 14 is laying down, sitting up, running, coughing, spasming, in respiratory distress, etc. The motion data can also be used to detect a breathing rate so as to allow a comparison between the breathing rate and the ECG information. Another use for the motion data is to detect abnormal motion associated with a disconnection of the sensor assembly 100 from electrode 4. These examples and many other applications are possible by knowing the relative movement of the electrode 4 in conjunction with the signal generated therefrom.

For instance, respiratory distress, both major and minor, can be correlated with ECG rhythm changes utilizing the simultaneous ECG signal recording and the sensor assembly 100 movement.

In addition, the system may allow sleep quality monitoring and related cardiovascular morbidity by correlating ECG data, respiration data and ECG electrode movement with abnormal/disruptive sleep by analyzing the motion of individual ECG electrode snaps attached to the patent.

Further, the system can also be used to acquire simultaneous patient activity and ECG while the user performs athletic training, competition, while the patient undergoes cardiac stress testing, while the patient undergoes physical therapy, or rehabilitation as an aid to performance enhancement or improvement, and/or for use in screening athletes for cardiovascular risks. The system can also generally provide a method for consumers to better monitor their health and/or exercise goals by allowing simultaneous acquisition of the ECG signal and movement/activity levels.

By integrating the accelerometer 3 with the snap electrode connector 6, it allows standard electrodes 4 to be used. As noted above, if it is desired, a separate motion analyzer can be used along with standard ECG equipment, so as to allow the benefits of the motion information without the necessity to upgrade/replace current ECG hardware. Indeed, the motion/acceleration data can be sent to a processor such as a personal computer (PC), a phone, etc., and the ECG data can be sent from the ECG equipment to the PC (or sent to the PC in parallel with the ECG equipment). Thus, all of the above data may be collected and analyzed using a PC and standard ECG equipment. This can reduce the costs of upgrading to the new ECG sensor system by not having to replace standard the ECG equipment.

While FIG. 7 illustrates electrodes 4 being placed on the torso of a patient 14, applications of the invention are not limited to the torso. Indeed, any portion of the patients body can have an electrode 4 and a sensor assembly 100 applied thereto. For instance, the electrodes 4 and sensor assemblies 100 may be applied to the arms, legs, back, head, lower abdomen, etc. The pattern and number of electrodes 4 and sensor assemblies 100 used is not particularly limited. Thus, the number and pattern of sensor assemblies 100 can be determined based on the level of detail and the information desired.

Indeed, the sensor assembly 100 could be used in conjunction with electroencephalography (EEG) or other electrical detection methods. It is possible to use the combined sensor assembly 100 to detect information other than simple electrical data from an electrode 4 (e.g., temperature from an electrode/sensor attached to a patient, etc.).

Further, while exemplary embodiments of sensor assembly 100 have been described as including a standard snap electrode connector 6, the invention is not limited to such. Indeed, the sensor assembly 100 may have the electrode 4 formed integrally therein. Also, it is contemplated that the electrode connector 6 may have a different fastening method (e.g., slide in, screw, have a male adaptor, etc.). This may be advantageous if the sensor assembly 100 is integrated into a garment or some wearable accessory, or is part of a bed or other object the patient 14 would interact with.

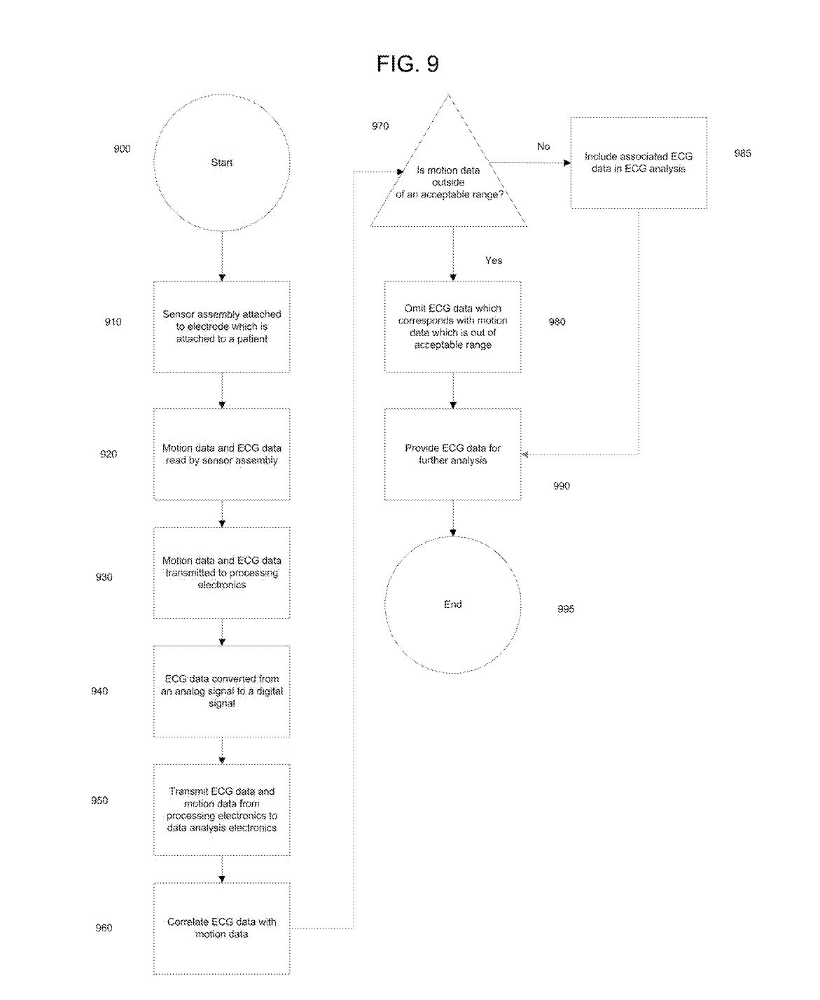

A skilled artisan will note that one or more of the aspects of the present invention may be performed on a computing device. The skilled artisan will also note that a computing device may be understood to be any device having a processor, memory unit, input, and output. This may include, but is not intended to be limited to, cellular phones, smart phones, tablet computers, laptop computers, desktop computers, personal digital assistants, etc. FIG. 8 illustrates a model computing device in the form of a computer 810, which is capable of performing one or more computer-implemented steps in practicing the method aspects of the present invention. Components of the computer 810 may include, but are not limited to, a processing unit 820, a system memory 830, and a system bus 821 that couples various system components including the system memory to the processing unit 820. The system bus 821 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI).

The computer 810 may also include a cryptographic unit 825. Briefly, the cryptographic unit 825 has a calculation function that may be used to verify digital signatures, calculate hashes, digitally sign hash values, and encrypt or decrypt data. The cryptographic unit 825 may also have a protected memory for storing keys and other secret data. In other embodiments, the functions of the cryptographic unit may be instantiated in software and run via the operating system.

A computer 810 typically includes a variety of computer readable media. Computer readable media can be any available media that can be accessed by a computer 810 and includes both volatile and nonvolatile media, removable and non-removable media. By way of example, and not limitation, computer readable media may include computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer 810. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

The system memory 830 includes computer storage media in the form of volatile and/or nonvolatile memory such as read only memory (ROM) 831 and random access memory (RAM) 832. A basic input/output system 833 (BIOS), containing the basic routines that help to transfer information between elements within computer 810, such as during start-up, is typically stored in ROM 831. RAM 832 typically contains data and/or program modules that are immediately accessible to and/or presently being operated on by processing unit 820. By way of example, and not limitation, FIG. 8 illustrates an operating system (OS) 834, application programs 835, other program modules 836, and program data 837.

The computer 810 may also include other removable/non-removable, volatile/nonvolatile computer storage media. By way of example only, FIG. 8 illustrates a hard disk drive 841 that reads from or writes to non-removable, nonvolatile magnetic media, a magnetic disk drive 851 that reads from or writes to a removable, nonvolatile magnetic disk 852, and an optical disk drive 855 that reads from or writes to a removable, nonvolatile optical disk 856 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 841 is typically connected to the system bus 821 through a non-removable memory interface such as interface 840, and magnetic disk drive 851 and optical disk drive 855 are typically connected to the system bus 821 by a removable memory interface, such as interface 850.

The drives, and their associated computer storage media discussed above and illustrated in FIG. 8, provide storage of computer readable instructions, data structures, program modules and other data for the computer 810. In FIG. 8, for example, hard disk drive 841 is illustrated as storing an OS 844, application programs 845, other program modules 846, and program data 847. Note that these components can either be the same as or different from OS 833, application programs 833, other program modules 836, and program data 837. The OS 844, application programs 845, other program modules 846, and program data 847 are given different numbers here to illustrate that, at a minimum, they may be different copies. A user may enter commands and information into the computer 810 through input devices such as a keyboard 862 and cursor control device 861, commonly referred to as a mouse, trackball or touch pad. Other input devices (not shown) may include a microphone, joystick, game pad, satellite dish, scanner, or the like. These and other input devices are often connected to the processing unit 820 through a user input interface 860 that is coupled to the system bus, but may be connected by other interface and bus structures, such as a parallel port, game port or a universal serial bus (USB). A monitor 891 or other type of display device is also connected to the system bus 821 via an interface, such as a graphics controller 890. In addition to the monitor, computers may also include other peripheral output devices such as speakers 897 and printer 896, which may be connected through an output peripheral interface 895.

The computer 810 may operate in a networked environment using logical connections to one or more remote computers, such as a remote computer 880. The remote computer 880 may be a personal computer, a server, a router, a network PC, a peer device or other common network node, and typically includes many or all of the elements described above relative to the computer 810, although only a memory storage device 881 has been illustrated in FIG. 8. The logical connections depicted in FIG. 8 include a local area network (LAN) 871 and a wide area network (WAN) 873, but may also include other networks 140. Such networking environments are commonplace in offices, enterprise-wide computer networks, intranets and the Internet.

When used in a LAN networking environment, the computer 810 is connected to the LAN 871 through a network interface or adapter 870. When used in a WAN networking environment, the computer 810 typically includes a modem 872 or other means for establishing communications over the WAN 873, such as the Internet. The modem 872, which may be internal or external, may be connected to the system bus 821 via the user input interface 860, or other appropriate mechanism. In a networked environment, program modules depicted relative to the computer 810, or portions thereof, may be stored in the remote memory storage device. By way of example, and not limitation, FIG. 8 illustrates remote application programs 885 as residing on memory device 881.

The communications connections 870 and 872 allow the device to communicate with other devices. The communications connections 870 and 872 are an example of communication media. The communication media typically embodies computer readable instructions, data structures, program modules or other data in a modulated data signal such as a carrier wave or other transport mechanism and includes any information delivery media. A “modulated data signal” may be a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Computer readable media may include both storage media and communication media.

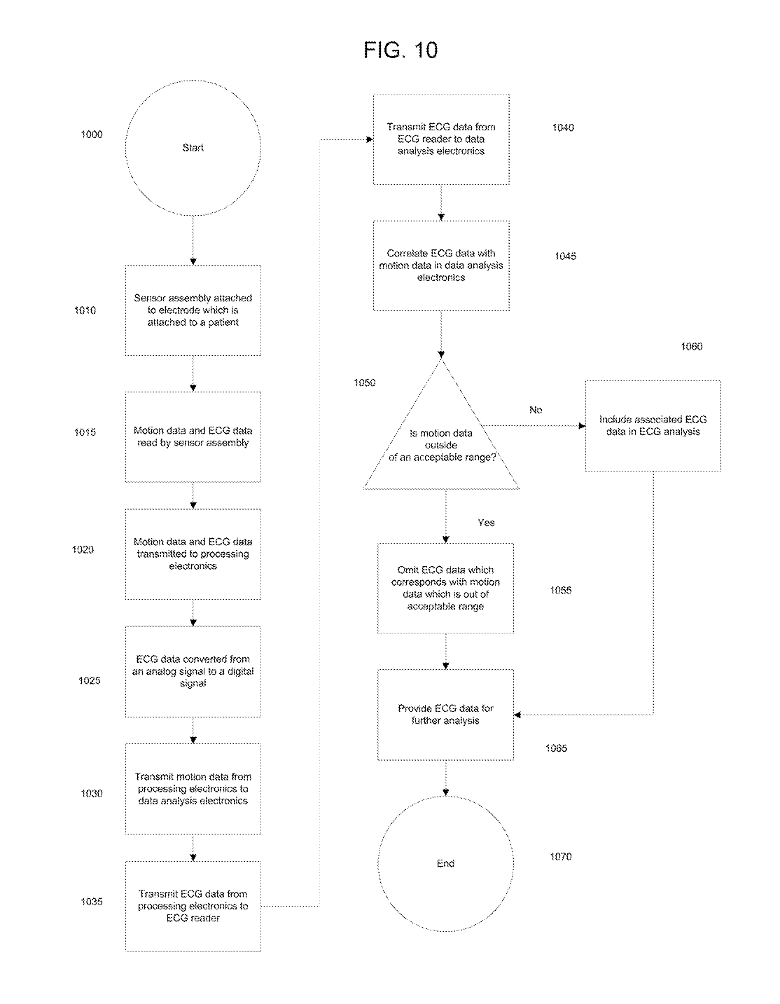

FIG. 9 illustrates an exemplary method of operation of the sensor assembly and related systems. The method starts at block 900. At block 910, the sensor assembly is attached to an electrode, the electrode being attached to a patient. At block 920, motion data from the accelerometer and ECG data from the electrode are read by the sensor assembly. At block 930, the motion data and ECG data is transferred to the processing electronics. At block 940, the ECG data is converted from an analog signal to a digital signal. Optionally, the ECG data may be amplified prior to conversion from an analog signal to a digital signal. At block 950, the ECG data and motion data are transmitted from the processing electronics to the data analysis electronics. This transmission may be done wirelessly. At block 960, the ECG data is correlated with the motion data. At block 970, it is determined whether the motion data is outside of an acceptable range. If so, at block 980, the ECG data associated with the out of range motion data is omitted. If the motion data is in an acceptable range, then the associated ECG data is included in the ECG data at block 985. The adjusted ECG data is then provided for further analysis at block 990. The further analysis may include displaying the ECG data, doing further processing of the data, transmitting the data, etc. The method ends at block 995.

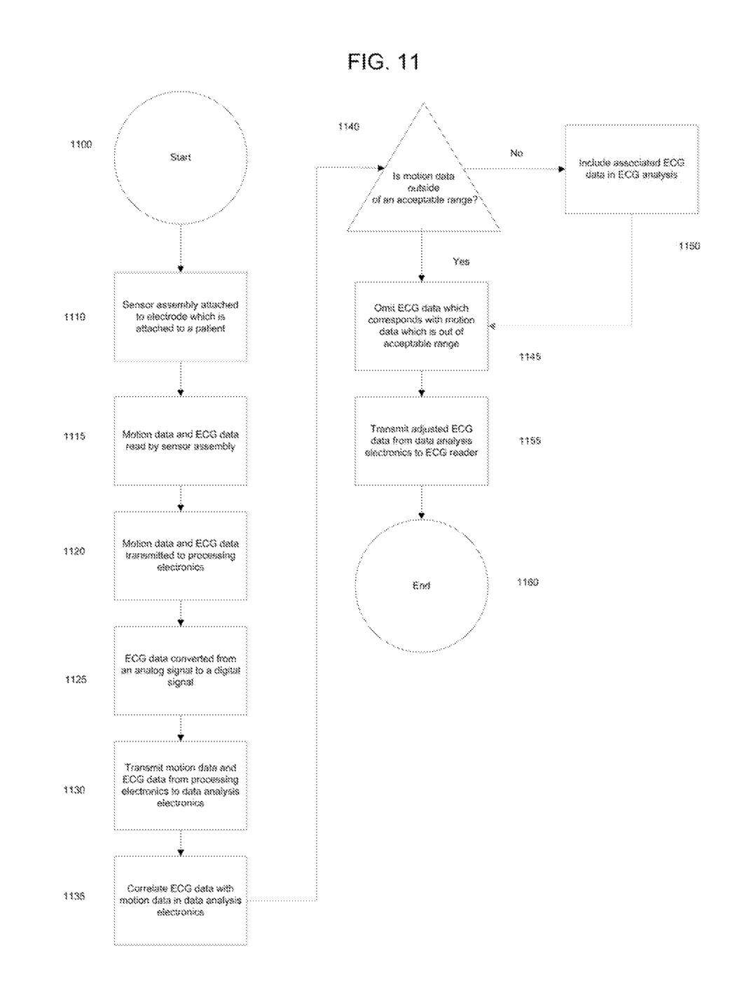

FIG. 10 illustrates another exemplary method of operation of the sensor assembly and related systems. The method starts at block 1000. At block 1010, the sensor assembly is attached to an electrode, the electrode being attached to a patient. At block 1015, motion data from the accelerometer and ECG data from the electrode are read by the sensor assembly. At block 1020, the motion data and ECG data are transferred to the processing electronics. At block 1025, the ECG data is converted from an analog signal to a digital signal. Optionally, the ECG data may be amplified prior to conversion from an analog signal to a digital signal. At block 1030, the motion data is transmitted from the processing electronics to the data analysis electronics. This transmission may be done wirelessly. At block 1035, the ECG data is transmitted from the processing electronics to an ECG reader. This transmission may be done wirelessly. At block 1040, the ECG data is transferred from the ECG reader to the data analysis electronics. At block 1045, the ECG data is correlated with the motion data. At block 1050, it is determined whether the motion data is outside of an acceptable range. If so, at block 1055, the ECG data associated with the out of range motion data is omitted. If the motion data is in an acceptable range, then the associated ECG data is included in the ECG data at block 1560. The adjusted ECG data is then provided for further analysis at block 1065. The further analysis may include displaying the ECG data, doing further processing of the data, transmitting the data, etc. The method ends at block 1070.

FIG. 11 illustrates another exemplary method of operation of the sensor assembly and related systems. The method starts at block 1100. At block 1110, the sensor assembly is attached to an electrode, the electrode being attached to a patient. At block 1115, motion data from the accelerometer and ECG data from the electrode are read by the sensor assembly. At block 1120, the motion data and ECG data are transferred to the processing electronics. At block 1125, the ECG data is converted from an analog signal to a digital signal. Optionally, the ECG data may be amplified prior to conversion from an analog signal to a digital signal. At block 1130, the motion data and the ECG data is transmitted from the processing electronics to the data analysis electronics. This transmission may be done wirelessly. At block 1135, the ECG data is correlated with the motion data. At block 1140, it is determined whether the motion data is outside of an acceptable range. If so, at block 1145, the ECG data associated with the out of range motion data is omitted. If the motion data is in an acceptable range, then the associated ECG data is included in the ECG data at block 1150. At block 1155, the adjusted ECG data is transmitted from the data analysis electronics to an ECG reader. This transmission may be done wirelessly. The method ends at block 1160.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed is:

1. A connector comprising:
a housing;
a female snap connector member carried by the housing and configured to mechanically and electrically connect to a male snap connector member of an electrode;
a three-axis accelerometer carried by the housing and configured to sense proper acceleration of the connector; and
a microprocessor in electrical communication with the snap connector and with the accelerometer;
wherein the microprocessor is configured to receive cardiac activity data from the electrode, to receive proper acceleration data from the accelerometer, and to correlate the cardiac activity data to the proper acceleration data to define processed data.

2. The connector according to claim 1 further comprising a computer readable non-transitory storage medium carried by the housing; wherein the microprocessor is configured to store at least one of the cardiac activity data, the proper acceleration data, and the processed data to the storage medium.

3. The connector according to claim 1 further comprising a lead wire at least partially carried by the housing and in data communication with the microprocessor and with a computing system, wherein the lead wire is configured to electronically convey at least one of the cardiac activity data, proper acceleration data, and the processed data to the computing system.

4. The connector according to claim 1 further comprising a wireless transmitter and in data communication with the microprocessor; wherein the wireless transmitter is configured to wirelessly transmit at least one of the cardiac activity data, proper acceleration data, and the processed data to a wireless receiver; and wherein the wireless receiver is configured to electronically convey at least one of the cardiac activity data, proper acceleration data, and the processed data to a computing system.

5. The connector according to claim 1 further comprising an amplifier and an analog-to-digital converter both; wherein the snap connector is configured to pass the cardiac activity data in analog format to the amplifier; wherein the amplifier is configured to pass the cardiac activity data in amplified form to the analog-to-digital converter; and wherein the analog-to-digital converter is configured to pass the cardiac activity data in digital format to the microprocessor.

6. The connector according to claim 1 wherein the accelerometer is configured to pass the proper acceleration data in digital format to the microprocessor.

7. The connector according to claim 1 wherein the electrode is an Association for the Advancement of Medical Instrumentation (AAMI) and/or an American National Standards Institute (ANSI) standard disposable electrocardiogram (ECG) electrode.

8. The connector according to claim 1 wherein the accelerometer is of a capacitive type.

9. A retrofit heart rate monitoring system comprising:
a computing system; and
at least one monitoring device in data communication with the computing system, and comprising:
an electrode comprising an Association for the Advancement of Medical Instrumentation (AAMI) and/or an American National Standards Institute (ANSI) standard male snap connector member;
a connector comprising:
a housing;
a female snap connector member carried by the housing and configured to mechanically and electrically connect to the male snap connector member of the electrode;
a three-axis accelerometer carried by the housing and configured to sense proper acceleration of the connector; and
a microprocessor in electrical communication with the snap connector and with the accelerometer, the microprocessor configured to receive cardiac activity data from the electrode, to receive proper acceleration data from the accelerometer, and to correlate the cardiac activity data to the proper acceleration data to define processed data;
wherein the cardiac activity data, proper acceleration data, and/or the processed data are transmitted by the at least one monitoring device to the computing system.

10. The system according to claim 9 wherein the connector further comprises a computer readable non-transitory storage medium; and wherein the microprocessor is configured to store at least one of the cardiac activity data, the proper acceleration data, and the processed data to the storage medium.

11. The system according to claim 9 wherein the connector further comprises a lead wire at least partially carried by the housing and in data communication with the microprocessor and with the computing system, wherein the lead wire is configured to electronically convey at least one of the cardiac activity data, proper acceleration data, and the processed data to the computing system.

12. The system according to claim 9 wherein the connector further comprises a wireless transmitter in data communication with the microprocessor; wherein the wireless transmitter is configured to wirelessly transmit at least one of the cardiac activity data, proper acceleration data, and the processed data to a wireless receiver; and wherein the wireless receiver is configured to electronically convey at least one of the cardiac activity data, proper acceleration data, and the processed data to the computing system.

13. The system according to claim 9 wherein the connector further comprises an amplifier and an analog-to-digital converter; wherein the snap connector is configured to pass the cardiac activity data in analog format to the amplifier; wherein the amplifier is configured to pass the cardiac activity data in amplified form to the analog-to-digital converter; and wherein the analog-to-digital converter is configured to pass the cardiac activity data in digital format to the microprocessor.

14. The system according to claim 9 wherein the accelerometer of the connector is configured to pass the proper acceleration data in digital format to the microprocessor.

15. The system according to claim 9 wherein the computing system is configured to determine whether the connector is detached from the electrode using at least one of the proper acceleration data and the processed data.

16. The system according to claim 9 wherein the computing system is configured to reduce movement artifact using the processed data.

17. The system of claim 9 wherein the computing system is configured to detect at least one of respiratory distress, sleep disturbance, and cardiovascular morbidity using the processed data.

18. A method of retrofitting an electrocardiogram (ECG) monitoring system using a connector comprising a female snap connector member, a three-axis accelerometer, and a microprocessor in electrical communication with the snap connector and with the accelerometer, the method comprising the steps of:
mechanically and electrically connecting the female snap connector member of the connector to a male snap connector member of an electrode;
detecting cardiac activity of a patient using the electrode;
detecting proper acceleration of the connector using the accelerometer;
transmitting cardiac activity data to the microprocessor from the electrode;
transmitting proper acceleration data to the microprocessor from the accelerometer; and
correlating, using the microprocessor, the cardiac activity data and the proper acceleration data to define processed data.

19. The method according to claim 18 wherein the connector further comprises a data communications channel comprising at least one of a lead wire and a wireless transmitter/receiver pair; wherein the method further comprises the step of conveying, using the data communications channel, at least one of the cardiac activity data, proper acceleration data, and the processed data from the connector to a computing system.

20. The method according to claim 18 further comprising the step of conveying the cardiac activity data to the electrocardiogram (ECG) monitoring system.

21. The method according to claim 18 further comprising the step of determining, using at least one of the proper acceleration data and processed data, if the connector is detached from the electrode.

22. The method according to claim 21 further comprising the step of providing an indication that the connector is detached from the electrode.

23. The method according to claim 18 further comprising the step of reducing movement artifact using the processed data.

24. The method according to claim 18 further comprising the step of detecting at least one of respiratory distress, sleep disturbance, and cardiovascular morbidity using the processed data.
